SEQUENCE LISTING (27 sequences)

SEQ ID NO: 1 - protein, 261 aa, Homo sapiens
  misc_feature (1)..(261): Prostate-specific antigen
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 649 aa, Homo sapiens
  misc_feature (1)..(649): Drebrin
MAGVSFSGHRLELLAAYEEVIREESAADWALYTYEDGSDDLKLAASGEGGLQELSGHFENQKVMYGFCSVKDSQAALPKYVLINWVGEDVPDARKCACASHVAKVAEFFQGVDVIVNASSVEDIDAGAIGQRLSNGLARLSSPVLHRLRLREDENAEPVGTTYQKTDAAVEMKRINREQFWEQAKKEEELRKEEERKKALDERLRFEQERMEQERQEQEERERRYREREQQIEEHRRKQQTLEAEEAKRRLKEQSIFGDHRDEEEETHMKKSESEVEEAAAIIAQRPDNPREFFKQQERVASASAGSCDVPSPFNHRPGSHLDSHRRMAPTPIPTRSPSDSSTASTPVAEQIERALDEVTSSQPPPLPPPPPPAQETQEPSPILDSEETRAAAPQAWAGPMEEPPQAQAPPRGPGSPAEDLMFMESAEQAVLAAPVEPATADATEIHDAADTIETDTATADTTVANNVPPAATSLIDLWPGNGEGASTLQGEPRAPTPPSGTEVTLAEVPLLDEVAPEPLLPAGEGCATLLNFDELPEPPATFCDPEEVEGESLAAPQTPTLPSALEELEQEQEPEPHLLTNGETTQKEGTQASEGYFSQSQEEEFAQSEELCAKAPPPVFYNKPPEIDITCWDADPVPEEEEGFEGGD

SEQ ID NO: 3 - protein, 393 aa, Homo sapiens [sequence omitted]

SEQ ID NO: 4 - protein, 1333 aa, Homo sapiens [sequence omitted]

SEQ ID NO: 5 - protein, 408 aa, Homo sapiens
  misc_feature (1)..(408): Glyceraldehyde-3-phosphate dehydrogenase, testis-specific
MSKRDIVLTNVTVVQLLRQPCPVTRAPPPPEPKAEVEPQPQPEPTPVREEIKPPPPPLPPHPATPPPKMVSVARELTVGINGFGRIGRLVLRACMEKGVKVVAVNDPFIDPEYMVYMFKYDSTHGRYKGSVEFRNGQLVVDNHEISVYQCKEPKQIPWRAVGSPYVVESTGVYLSIQAASDHISAGAQRVVISAPSPDAPMFVMGVNENDYNPGSMNIVSNASCTTNCLAPLAKVIHERFGIVEGLMTTVHSYTATQKTVDGPSRKAWRDGRGAHQNIIPASTGAAKAVTKVIPELKGKLTGMAFRVPTPDVSVVDLTCRLAQPAPYSAIKEAVKAAAKGPMAGILAYTEDEVVSTDFLGDTHSSIFDAKAGIALNDNFVKLISWYDNEYGYSHRVVDLLRYMFSRDK

SEQ ID NO: 6 - protein, 793 aa, Homo sapiens
  misc_feature (1)..(793): Caldesmon
MDDFERRRELRRQKREEMRLEAERIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQVEVMVEEKTTESQEETVVMSLKNGQISSEEPKQEEEREQGSDEISHHEKMEEEDKERAEAERARLEAEERERIKAEQDKKIADERARIEAEEKAAAQERERREAEERERMREEEKRAAEERQRIKEEEKRAAEERQRIKEEEKRAAEERQRIKEEEKRAAEERQRARAEEEEKAKVEEQKRNKQLEEKKRAMQETKIKGEKVEQKIEGKWVNEKKAQEDKLQTAVLKKQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 7 - protein, 641 aa, Homo sapiens
  misc_feature (1)..(641): Heat shock 70 kDa protein 1A/1B
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 8 - protein, 295 aa, Homo sapiens
  misc_feature (1)..(295): G1/S-specific cyclin-D1
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 9 - protein, 445 aa, Homo sapiens
  misc_feature (1)..(445): Tubulin beta-2A chain
MREIVHIQAGQCGNQIGAKFWEVISDEHGIDPTGSYHGDSDLQLERINVYYNEAAGNKYVPRAILVDLEPGTMDSVRSGPFGQIFRPDNFVFGQSGAGNNWAKGHYTEGAELVDSVLDVVRKESESCDCLQGFQLTHSLGGGTGSGMGTLLISKIREEYPDRIMNTFSVMPSPKVSDTVVEPYNATLSVHQLVENTDETYSIDNEALYDICFRTLKLTTPTYGDLNHLVSATMSGVTTCLRFPGQLNADLRKLAVNMVPFPRLHFFMPGFAPLTSRGSQQYRALTVPELTQQMFDSKNMMAACDPRHGRYLTVAAIFRGRMSMKEVDEQMLNVQNKNSSYFVEWIPNNVKTAVCDIPPRGLKMSATFIGNSTAIQELFKRISEQFTAMFRRKAFLHWYTGEGMDEMEFTEAESNMNDLVSEYQQYQDATADEQGEFEEEEGEDEA

SEQ ID NO: 10 - protein, 765 aa, Homo sapiens
  misc_feature (1)..(765): DNA topoisomerase 1
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 11 - protein, 919 aa, Homo sapiens
  misc_feature (1)..(919): Androgen receptor
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 12 - protein, 479 aa, Homo sapiens
  misc_feature (1)..(479): Caspase-8
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 13 - protein, 432 aa, Homo sapiens
  misc_feature (1)..(432): Cyclin-A2
MLGNSAPGPATREAGSALLALQQTALQEDQENINPEKAAPVQQPRTRAALAVLKSGNPRGLAQQQRPKTRRVAPLKDLPVNDEHVTVPPWKANSKQPAFTIHVDEAEKEAQKKPAESQKIEREDALAFNSAISLPGPRKPLVPLDYPMDGSFESPHTMDMSIILEDEKPVSVNEVPDYHEDIHTYLREMEVKCKPKVGYMKKQPDITNSMRAILVDWLVEVGEEYKLQNETLHLAVNYIDRFLSSMSVLRGKLQLVGTAAMLLASKFEEIYPPEVAEFVYITDDTYTKKQVLRMEHLVLKVLTFDLAAPTVNQFLTQYFLHQQPANCKVESLAMFLGELSLIDADPYLKYLPSVIAGAAFHLALYTVTGQSWPESLIRKTGYTLESLKPCLMDLHQTYLKAPQHAQQSIREKYKNSKYHGVSLLNPPETLNL

SEQ ID NO: 14 - protein, 189 aa, Homo sapiens
  misc_feature (1)..(189): Protein DJ-1
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 15 - protein, 326 aa, Homo sapiens
  misc_feature (1)..(326): Peroxisome biogenesis factor 10
MAPAAASPPEVIRAAQKDEYYRGGLRSAAGGALHSLAGARKWLEWRKEVELLSDVAYFGLTTLAGYQTLGEEYVSIIQVDPSRIHVPSSLRRGVLVTLHAVLPYLLDKALLPLEQELQADPDSGRPLQGSLGPGGRGCSGARRWMRHHTATLTEQQRRALLRAVFVLRQGLACLQRLHVAWFYIHGVFYHLAKRLTGITYLRVRSLPGEDLRARVSYRLLGVISLLHLVLSMGLQLYGFRQRQRARKEWRLHRGLSHRRASLEERAVSRNPLCTLCLEERRHPTATPCGHLFCWECITAWCSSKAECPLCREKFPPQKLIYLRHYR

SEQ ID NO: 16 - protein, 530 aa, Homo sapiens
  misc_feature (1)..(530): PC4 and SFRS1-interacting protein
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEEKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 17 - protein, 403 aa, Homo sapiens [sequence omitted]

SEQ ID NO: 18 - protein, 591 aa, Homo sapiens
  misc_feature (1)..(591): Paxillin
MDDLDALLADLESTTSHISKRPVFLSEETPYSYPTGNHTYQEIAVPPPVPPPPSSEALNGTILDPLDQWQPSSSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKIQGLEQRADGERCWAAGWPRDGGRSSPGGQDEGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 19 - protein, 414 aa, Homo sapiens
  misc_feature (1)..(414): TAR DNA-binding protein 43
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 20 - protein, 2541 aa, Homo sapiens
  misc_feature (1)..(2541): Talin-1
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

SEQ ID NO: 21 - protein, 2000 aa, Homo sapiens
  misc_feature (1)..(2000): Chromodomain-helicase-DNA-binding protein 3
MKAADTVILWARSKNDQLRISFPPGLCWGDRMPDKDDIRLLPSALGVKKRKRGPKKQKENKPGKPRKRKKRDSEEEFGSERDEYREKSESGGSEYGTGPGRKRRRKHREKKEKKTKRRKKGEGDGGQKQVEQKSSATLLLTWGLEDVEHVFSEEDYHTLTNYKAFSQFMRPLIAKKNPKIPMSKMMTILGAKWREFSANNPFKGSAAAVAAAAAAAAAAVAEQVSAAVSSATPIAPSGPPALPPPPAADIQPPPIRRAKTKEGKGPGHKRRSKSPRVPDGRKKLRGKKMAPLKIKLGLLGGKRKKGGSYVFQSDEGPEPEAEESDLDSGSVHSASGRPDGPVRTKKLKRGRPGRKKKKVLGCPAVAGEEEVDGYETDHQDYCEVCQQGGEIILCDTCPRAYHLVCLDPELDRAPEGKWSCPHCEKEGVQWEAKEEEEEYEEEGEEEGEKEEEDDHMEYCRVCKDGGELLCCDACISSYHIHCLNPPLPDIPNGEWLCPRCTCPVLKGRVQKILHWRWGEPPVAVPAPQQADGNPDVPPPRPLQGRSEREFFVKWVGLSYWHCSWAKELQLEIFHLVMYRNYQRKNDMDEPPPLDYGSGEDDGKSDKRKVKDPHYAEMEEKYYRFGIKPEWMTVHRIINHSVDKKGNYHYLVKWRDLPYDQSTWEEDEMNIPEYEEHKQSYWRHRELIMGEDPAQPRKYKKKKKELQGDGPPSSPTNDPTVKYETQPRFITATGGTLHMYQLEGLNWLRFSWAQGTDTILADEMGLGKTIQTIVFLYSLYKEGHTKGPFLVSAPLSTIINWEREFQMWAPKFYVVTYTGDKDSRAIIRENEFSFEDNAIKGGKKAFKMKREAQVKFHVLLTSYELITIDQAALGSIRWACLVVDEAHRLKNNQSKFFRVLNGYKIDHKLLLTGTPLQNNLEELFHLLNFLTPERFNNLEGFLEEFADISKEDQIKKLHDLLGPHMLRRLKADVFKNMPAKTELIVRVELSPMQKKYYKYILTRNFEALNSRGGGNQVSLLNIMMDLKKCCNHPYLFPVAAMESPKLPSGAYEGGALIKSSGKLMLLQKMLRKLKEQGHRVLIFSQMTKMLDLLEDFLDYEGYKYERIDGGITGALRQEAIDRFNAPGAQQFCFLLSTRAGGLGINLATADTVIIFDSDWNPHNDIQAFSRAHRIGQANKVMIYRFVTRASVEERITQVAKRKMMLTHLVVRPGLGSKAGSMSKQELDDILKFGTEELFKDENEGENKEEDSSVIHYDNEAIARLLDRNQDATEDTDVQNMNEYLSSFKVAQYVVREEDKIEEIEREIIKQEENVDPDYWEKLLRHHYEQQQEDLARNLGKGKRVRKQVNYNDAAQEDQDNQSEYSVGSEEEDEDFDERPEGRRQSKRQLRNEKDKPLPPLLARVGGNIEVLGFNTRQRKAFLNAVMRWGMPPQDAFTTQWLVRDLRGKTEKEFKAYVSLFMRHLCEPGADGSETFADGVPREGLSRQQVLTRIGVMSLVKKKVQEFEHINGRWSMPELMPDPSADSKRSSRASSPTKTSPTTPEASATNSPCTSKPATPAPSEKGEGIRTPLEKEEAENQEEKPEKNSRIGEKMETEADAPSPAPSLGERLEPRKIPLEDEVPGVPGEMEPEPGYRGDREKSATESTPGERGEEKPLDGQEHRERPEGETGDLGKREDVKGDRELRPGPRDEPRSNGRREEKTEKPRFMFNIADGGFTELHTLWQNEERAAISSGKLNEIWHRRHDYWLLAGIVLHGYARWQDIQNDAQFAIINEPFKTEANKGNFLEMKNKFLARRFKLLEQALVIEEQLRRAAYLNLSQEPAHPAMALHARFAEAECLAESHQHLSKESLAGNKPANAVLHKVLNQLEELLSDMKADVTRLPATLSRIPPIAARLQMSERSILSRLASKGTEPHPTPAYPPGPYATPPGYGAAFSAAPVGALAAAGANYSQMPAGSFITAATNGPPVLVKKEKEMVGALVSDGLDRKEPRAGEVICIDD

SEQ ID NO: 22 - protein, 925 aa, Homo sapiens
  misc_feature (1)..(925): Nuclear factor of activated T-cells, cytoplasmic 2
MNAPERQPQPDGGDAPGHEPGGSPQDELDFSILFDYEYLNPNEEEPNAHKVASPPSGPAYPDDVLDYGLKPYSPLASLSGEPPGRFGEPDRVGPQKFLSAAKPAGASGLSPRIEITPSHELIQAVGPLRMRDAGLLVEQPPLAGVAASPRFTLPVPGFEGYREPLCLSPASSGSSASFISDTFSPYTSPCVSPNNGGPDDLCPQFQNIPAHYSPRTSPIMSPRTSLAEDSCLGRHSPVPRPASRSSSPGAKRRHSCAEALVALPPGASPQRSRSPSPQPSSHVAPQDHGSPAGYPPVAGSAVIMDALNSLATDSPCGIPPKMWKTSPDPSPVSAAPSKAGLPRHIYPAVEFLGPCEQGERRNSAPESILLVPPTWPKPLVPAIPICSIPVTASLPPLEWPLSSQSGSYELRIEVQPKPHHRAHYETEGSRGAVKAPTGGHPVVQLHGYMENKPLGLQIFIGTADERILKPHAFYQVHRITGKTVTTTSYEKIVGNTKVLEIPLEPKNNMRATIDCAGILKLRNADIELRKGETDIGRKNTRVRLVFRVHIPESSGRIVSLQTASNPIECSQRSAHELPMVERQDTDSCLVYGGQQMILTGQNFTSESKVVFTEKTTDGQQIWEMEATVDKDKSQPNMLFVEIPEYRNKHIRTPVKVNFYVINGKRKRSQPQHFTYHPVPAIKTEPTDEYDPTLICSPTHGGLGSQPYYPQHPMVAESPSCLVATMAPCQQFRTGLSSPDARYQQQNPAAVLYQRSKSLSPSLLGYQQPALMAAPLSLADAHRSVLVHAGSQGQSSALLHPSPTNQQASPVIHYSPTNQQLRCGSHQEFQHIMYCENFAPGTTRPGPPPVSQGQRLSPGSYPTVIQQQNATSQRAAKNGPPVSDQKEVLPAGVTIKQEQNLDQTYLDDVNEIIRKEFSGPPARNQT

SEQ ID NO: 23 - protein, 255 aa, Homo sapiens
  misc_feature (1)..(255): Syntaxin-6
MSMEDPFFVVKGEVQKAVNTAQGLFQRWTELLQDPSTATREEIDWTTNELRNNLRSIEWDLEDLDETISIVEANPRKFNLDATELSIRKAFITSTRQVVRDMKDQMSTSSVQALAERKNRQALLGDSGSQNWSTGTTDKYGRLDRELQRANSHFIEEQQAQQQLIVEQQDEQLELVSGSIGVLKNMSQRIGGELEEQAVMLEDFSHELESTQSRLDNVMKKLAKVSHMTSDRRQWCAIAILFAVLLVVLILFLVL

SEQ ID NO: 24 - protein, 636 aa, Homo sapiens
  misc_feature (1)..(636): Tumor protein p73
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 25 - protein, 492 aa, Homo sapiens
  misc_feature (1)..(492): Heat shock factor protein 4
MQEAPAALPTEPGPSPVPAFLGKLWALVGDPGTDHLIRWSPSGTSFLVSDQSRFAKEVLPQYFKHSNMASFVRQLNMYGFRKVVSIEQGGLLRPERDHVEFQHPSFVRGREQLLERVRRKVPALRGDDGRWRPEDLGRLLGEVQALRGVQESTEARLRELRQQNEILWREVVTLRQSHGQQHRVIGKLIQCLFGPLQAGPSNAGGKRKLSLMLDEGSSCPTPAKFNTCPLPGALLQDPYFIQSPLPETNLGLSPHRARGPIISDIPEDSPSPEGTRLSPSSDGRREKGLALLKEEPASPGGDGEAGLALAPNECDFCVTAPPPLPVAVVQAILEGKGSFSPEGPRNAQQPEPGDPREIPDRGPLGLESGDRSPESLLPPMLLQPPQESVEPAGPLDVLGPSLQGREWTLMDLDMELSLMQPLVPERGEPELAVKGLNSPSPGKDPTLGAPLLLDVQAALGGPALGLPGALTIYSTPESRTASYLGPEASPSP

SEQ ID NO: 26 - protein, 851 aa, Homo sapiens
  misc_feature (1)..(851): Signal transducer and activator of transcription 2
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

SEQ ID NO: 27 - protein, 416 aa, Homo sapiens
  misc_feature (1)..(416): Prostate tumor-overexpressed gene 1 protein
MVRPRRAPYRSGAGGPLGGRGRPPRPLVVRAVRSRSWPASPRGPQPPRIRARSAPPMEGARVFGALGPIGPSSPGLTLGGLAVSEHRLSNKLLAWSGVLEWQEKRRPYSDSTAKLKRTLPCQAYVNQGENLETDQWPQKLIMQLIPQQLLTTLGPLFRNSQLAQFHFTNRDCDSLKGLCRIMGNGFAGCMLFPHISPCEVRVLMLLYSSKKKIFMGLIPYDQSGFVSAIRQVITTRKQAVGPGGVNSGPVQIVNNKFLAWSGVMEWQEPRPEPNSRSKRWLPSHVYVNQGEILRTEQWPRKLYMQLIPQQLLTTLVPLFRNSRLVQFHFTKDLETLKSLCRIMDNGFAGCVHFSYKASCEIRVLMLLYSSEKKIFIGLIPHDQGNFVNGIRRVIANQQQVLQRNLEQEQQQRGMGG

(19) United States
(12) Patent Application Publication
(10) Pub. No.: US 2014/0066325 A1
(43) Pub. Date: Mar. 6, 2014

(54) Protein Biomarkers for the Diagnosis of Prostate Cancer

(71) Applicants: Brian Liu, Somerville, MA (US); Robert Caiazzo, Essex, CT (US); David Ure, Wellesley, MA (US); James Nelson, Raleigh, NC (US)

(72) Inventors: Brian Liu, Somerville, MA (US); Robert Caiazzo, Essex, CT (US); David Ure, Wellesley, MA (US); James Nelson, Raleigh, NC (US)

(73) Assignee: THE BRIGHAM AND WOMEN'S HOSPITAL, INC., Boston, MA (US)

(21) Appl. No.: 14/005,551

(22) PCT Filed: Mar. 15, 2012
(86) PCT No.: PCT/US12/29195

§ 371 Date: Sep. 16, 2013

Related U.S. Application Data
(60) Provisional application No. 61/453,951, filed on Mar. 17, 2011.

(51) Int. Cl. G01N 33/574

(52) U.S. Cl. CPC G01N 33/57434
USPC 506/9; 436/501; 435/7.92; 506/18

(57) ABSTRACT

The invention is directed to tumor associated markers (TAMs) and autoantibody biomarkers that can be used diagnostically. It also includes methods for detection of the markers and compositions that can be used in carrying out assays

### CROSS REFERENCE TO RELATED APPLICATIONS

The present application claims the benefit of U.S. provisional application 61/453,951, filed on Mar. 17, 2011. This prior application is hereby incorporated by reference in its entirety.

### FIELD OF THE INVENTION

The present invention is directed to tumor associated markers (TAMs) and associated autoantibodies that can be used in diagnostics, particularly for diagnosing prostate cancer, and for distinguishing clinically significant forms of prostate cancer from benign prostate hyperplasia (BPH).

### BACKGROUND OF THE INVENTION

Prostate cancer is the most commonly diagnosed cancer in American men over the age of 50. Currently, the standard for detection of prostate cancer involves screening blood for levels of prostate specific antigen (PSA), digital-rectal examination, and needle biopsy of the prostate. However, PSA levels are compromised by variations in the amount of PSA produced by benign prostatic tissue (Brawer M K, CA Cancer J Clin 49:264-281 (1999)). Prostate biopsy should be considered in all patients who have nodules and/or PSA levels above 4 ng/mL. Benign prostate hyperplasia (BPH), however, can produce PSA at levels greater than 4 ng/mL. In addition, 25 to 35% of the men with organ-confined prostate cancer can present with PSA levels below 4 ng/mL. This underscores the fact that serum PSA alone is not a perfect marker for distinguishing prostate cancer from BPH (Brawer M K, CA Cancer J Clin 49:264-281 (1999)). Thus, there is a need to identify easily measurable biomarker(s) capable of ruling out a diagnosis of cancer for patients with non-malignant prostatic disease such as BPH, and reducing the need for unnecessary biopsies.

One strategy for improving diagnostic accuracy involves taking advantage of the body's own immune system. Proteins released by malignant cells but not normally present in serum may elicit a host immune response that generates an amplification of signal in the form of antibodies relative to the amount of the corresponding antigen. Thus, even small amounts of tumor-associated antigens (TAAs) may be identified based upon antibody levels, especially during early stages of cancer formation (Finn, N Engl J Med 353:1288-1290 (2005)).

Previous reports have described a microarray assay for examining the antibody profile of a sample of blood, plasma or serum (PCT/US2006/016543; US 2008/0081339; Qin, et al., Proteomics 6:3199-209 (2006); Ehrlich, et al., Nat. Protocols 1:452-60 (2006); Liu, et al., Expert Rev. Proteomics 3:283-96 (2006)). One advantage of this “reverse capture” autoantibody microarray is that it uses human cell lysate as the protein source for the detection of antigen-specific antibodies. Thus, immunogenic epitopes that may not be present in recombinant proteins are preserved (Qin, et al., Proteomics 6:3199-3209 (2006); Ehrlich, et al., Nature Protocols 1:452-460 (2006); Ehrlich, et al., Proteomics Clin. Appl. 1:476-485 (2007)). The assay has previously been used in connection with detecting markers associated with prostate cancer (US 2009/0075305; PCT/US2007/21112).

### SUMMARY DESCRIPTION OF THE INVENTION

Based on experiments using an initial set of over 500 cancer related antigens, supplemented with literature searches for cancer related antigens, a customized array containing 27 antigens was developed. Using this array with a reverse capture assay methodology and human cell lysate as a protein source, autoantibody profiles have been identified which may be used in diagnosing prostate cancer and in distinguishing prostate cancer from BPH in patients. Of the 27 antigens identified, 21 antigens (corresponding to the first 21 entries in Table 4) and their associated autoantibodies appear to be particularly useful at discriminating between prostate cancer and BPH.

Unless otherwise indicated expressly or by context, it will be understood that, although the term “biomarker” or “tumor associated marker” as used herein refers to a human protein, the protein may be identified by its name, by its amino acid sequence, or by the name of the gene that encodes it. The proteins serve as antigens that are recognized by autoantibodies. Table 7 has information that may be used to correlate antigen biomarkers to gene names and amino acid sequences.

Each antigen biomarker identified herein may be combined with one or more additional biomarker antigens to form a panel of antigens (a multiplexed diagnostic test). These panels can help in diagnosing prostate cancer, e.g., by discriminating between prostate cancer and BPH more accurately than a standard PSA test alone. It was calculated that a multiplexed platform consisting of 5 selected antigens could be used to detect prostate cancer in a high percentage of subjects (FIG. 4).

### Detailed Summary

In its first aspect, the invention is directed to a method of diagnostically evaluating a subject for prostate cancer by obtaining a “test” biological sample and assaying the sample for at least one, and preferably two or more, of the following tumor associated markers (TAMs): TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, and CCNA2. In a preferred embodiment, this is done by evaluating the sample for autoantibodies that recognize these TAMs. Autoantibody tests for 2 or more, and preferably 5 or more, of the first 21 of these markers will be of particular value in distinguishing between prostate cancer and BPH, e.g., in a patient that is suspected of having prostate cancer based on clinical criteria or an elevated PSA test performed using different methodology.

The results from the test biological sample are compared to those from one or more similar “control samples” obtained from subjects known to be disease free or to have benign prostate disease, e.g., benign prostate hyperplasia. Alternatively, concentration thresholds, or ratio's thereof can be used and compared with the results from the test samples. If the comparison indicates that the test sample has a higher amount of one or more (preferably 2 or more and more preferably 5 or more) TAMs and/or autoantibodies, this is an indication that the test subject has prostate cancer. In general, as the number of elevated TAMs and/or autoantibodies increases, so does the probability that prostate cancer is present.

Examples of test biological samples that can be used include blood, plasma, serum, urine, saliva, prostate tissue and prostate fluid (i.e., fluid immediately surrounding the prostate gland). The most preferred of these is blood, plasma or serum. The amount of TAMs and/or autoantibodies present in the biological sample can be determined by any method known in the art, e.g. by ELISA, antibody profiling assays, reverse capture assays, immunoassays, radioimmunoassay, radioreceptor assay, bead assays, or flow based assays. The preferred method is by an antibody profiling assay, optionally processed such that a number of temporally separated detection events are recorded to enable binding curves from the profiling assay to be constructed. For the purposes of the present application, the antibody profiling assay is defined as assessing the amount of TAM present indirectly by examining the amount of antibody against the TAM in the biological sample. General guidance regarding similar such assays may be found in PCT/US2006/016543.

In all cases, it is preferred that samples be assayed for at least 2 of the cancer specific TAMs, and more preferably at least 5. One of the identified biomarkers is PSA (listed as SEQ ID NO:1 in table 7). PSA may be included and assayed as part of a biomarker array, or separately using a different diagnostic assay. The 5 most preferred antigens for assay are: TARDBP, TLN1, PARK7, PSIP1, CALDI1 (FIG. 4). These may be used as a group in a single assay or together with other TAMs.

In a preferred embodiment, each TAM is attached to a support such as a plate, slide, chip or cartridge by a monoclonal antibody that specifically recognizes it. Although not preferred, each TAM may alternatively be attached directly to the support. The support with attached TAMs may be included as part of a kit along with instructions concerning its use in performing a diagnostic assay for prostate cancer. The kit may also optionally include a control sample derived from one or more individuals known not to have prostate disease or from one or more patients with benign prostate hyperplasia. The support may then be used in assays to help in diagnosing patients for prostate cancer.

The invention also includes an assay for comparing the antibodies present in samples of blood, plasma, serum, urine, saliva, prostate fluid or tissue. The assay involves obtaining an immobilized array of TAMs, each TAM being attached to the surface of a solid support by an antibody that specifically recognizes it. Alternatively, in a less preferred embodiment, the TAM may be directly attached to the support surface. The supports must include at least one, preferably at least two and, more preferably, at least five markers selected from: TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1 and CCNA2.

In a preferred embodiment, antibodies derived from a sample of blood, serum, plasma or other bodily fluid are detected using a secondary labeled antibody. In another embodiment, the antibodies are directly labeled to facilitate detection. In yet another embodiment, antibodies from a sample of blood, serum, plasma, or other bodily fluid are detected using a label-free detection method such as surface plasmon resonance, mass based detection, or other label free detection methods known to those skilled in the art.

Preferably, the labels used on antibodies or secondary antibodies are dyes or fluorescent labels, and the assay is processed in a ‘flow based platform’ to enable the construction of a binding curve for each test antibody.

### DESCRIPTION OF THE DRAWINGS

FIG. 1: Quality control (QC) gel image with normal and abnormal bands. FIG. 1 is an image of a QC gel used to test for sample IgGs purity. Two of the samples contain extraneous material which is shown as a third band at the bottom of the gel. These samples are omitted from the analysis.

FIG. 2: Array protocol scheme. This illustration depicts the protocol of the “reverse capture” slides with respect to where samples are placed, where duplicates are located, what concentrations are used, and whether cell lysate is present.

FIG. 3: Prostate cancer versus BPH array reactivity images. 3A and 3B show comparisons of a prostate cancer (PC) patient's array to a BPH patient's array. Within the arrays, the three replicates of TARDBP and TLN1 are circled showcasing their much greater fluorescence in the cancer samples. 3C is a layout map that defines the location of each spot in the array image. Table 3 lists the spot IDs for the layout.

FIG. 4: Antibodies with the five greatest AUC values and their corresponding ROC curves. FIG. 4 shows the five antibodies that have the greatest AUC values, and therefore greatest predictive value. TARDBP, TLN1, PARK7, PSIP1, and CALD1 each possess AUC values over 75% and when combined, have a positive predictive value of 95%.

FIG. 5: Reproducibility of the reverse capture array. FIG. 5 shows eight arrays, two from each prostate cancer patient. A high degree of reproducibility is seen for each patient's duplicate arrays and even between patients.

### DETAILED DESCRIPTION OF THE INVENTION

The present invention is based upon the identification of antigens and associated autoantibodies that can be used to identify patients with prostate cancer. These are shown in Table 4. Although an increase in any of these antigens (or their autoantibodies) in the serum of a subject is suggestive of the presence of prostate cancer, a better and more clinically relevant assessment can be made by examining two or more, and preferably 5 or more of the antigens or autoantibodies. One way of doing this is to use an ELISA, radioimmuno- or radioreceptor assay to examine individual antigens. However, it is preferred that microarray plates, slides, chips or cartridges be used to examine multiple antigens at once.

In a preferred embodiment, this is done by immobilizing an array of monoclonal antibodies, each recognizing a specific antigen, to a surface. Many plastic, glass, polymer based, metallic, nylon or other surfaces are known in the art and can be used for this purpose. Monoclonal antibodies appropriate for attachment are commercially available. If desired, fragments derived from the monoclonal antibodies that maintain the ability to specifically recognize antigen may also be used.

The antigens may optionally be attached to the immobilized antibodies by lysing cells derived from culture or in vivo, removing cellular debris and then incubating the crude antigen solution with the array of immobilized antibodies. At the end of the incubation, unattached materials and antigens are removed, thereby leaving behind an array of antigens attached to slides, plates, beads, chips, or cartridges by the immobilized monoclonal antibodies. The identity of each of the attached antigens is known from the specificity of the antibody to which it is attached. In other words, each antibody is at a specific location on the slide, plate, chips, or cartridges and recognizes only one particular type of antigen. In another embodiment, the array of immobilized antigens may be directly arrayed/printed onto a surface, or otherwise immobilized using methods known to those skilled in the art. Although less preferred than native antigens (antigens from cancer patient cells), recombinant antigens (laboratory developed antigens) may be used in assays.

Once the array of immobilized antigens has been prepared, the antibody samples that will undergo testing are then prepared. A sample of serum, plasma, blood, urine, saliva, prostate fluid or tissue is removed from a test subject being tested for prostate cancer (the test biological sample). The IgG fraction present in the samples is then optionally isolated using any method known in the art and the resulting antibodies are, in some embodiments labeled. Any type of label that can be detected using a microarray assay is compatible with the present invention, with fluorescent dyes such as Cy3 and Cy5 preferred. Optionally, the IgG fraction will not be directly labeled, instead, a secondary detection antibody or other labeled reagent will be used to enable detection of the biomarkers. In yet other embodiments, the detection of the biomarkers will be made using a label free approach, methodology or platform. In still others, the detection of the biomarkers will be made by any platform and method known to those skilled in the art. In addition, the detection of the biomarkers may be made directly from the sample of blood, serum, plasma, urine, saliva or prostate fluid or tissue without first isolating the IgG fraction present in the sample.

The results from the test biological sample may be compared to those from one or more similar “control samples” obtained from subjects known to be disease free or to have benign prostate disease, e.g., benign prostate hyperplasia. Alternatively, concentration thresholds, or ratio's thereof can be used as comparisons to the results from the test samples. If the comparison indicates that the test sample has a higher amount of one or more TAMs and/or autoantibodies; or reaches certain concentration thresholds or ratio's, this is an indication that the test subject has prostate cancer. In general, as the number of elevated TAMs and/or autoantibodies increases, so does the probability that prostate cancer is present.

Microarray plates or slides containing an array of two or more of the identified TAMs may be prepared and included as part of a kit. The kit will also include instructions describing how the plates or slides can be used in diagnostic assays for prostate cancer. In addition, it may include other components needed in assays such as buffers or a “control” preparation of antibodies.

Assays utilizing arrays of two or more of the TAMs in Table 4 may also be combined with assays of other factors of diagnostic value.

### EXAMPLES

The current study examines the differential expression of autoantibodies to native prostate tumor antigens by prostate cancer and benign prostatic hyperplasia (BPH) patients. The platform used in this research was the reverse capture autoantibody microarray described by Qin, et al. (Proteomics 6:3199-209 (2006)) and Ehrlich, et al. (Nat. Protocols 1:452-60 (2006))

### Materials and Methods

Cell Culture and Lysis

Native antigens used in the reverse capture platform were obtained from two human prostate cancer cell lines. Androgen-responsive LNCaP and androgen-independent PC-3 cells were initially obtained via the American Type Culture Collection in Rockville, Md. Cells were cultured in RPMI with L-glutamine (Invitrogen, Carlsbad, Calif.), 10% FBS, and 100 IU/mL penicillin and 100 mg/mL streptomycin. By scraping cells from plates and resolving the cell pellets in Protein Extraction/Labeling Buffer (Clontech Laboratories, Mountain View, Calif.), whole-cell extracts consisting of membrane-bound and cytosolic proteins were obtained. After inverting the suspension for 10 minutes at room temperature, the insoluble fraction was removed by 30 min centrifugation at 10,000 g at 4° C. After extracting the protein rich region, the protein concentrations were determined using a BCA Protein Assay Reagent kit according to the manufacturer's instructions (Pierce Biotechnology, Rockford, Ill.).

Serum Collection

Serum samples were collected for both benign prostate hyperplasia (BPH) patients and prostate cancer patients during routine pre-operative appointments according to an IRB approved protocol. Samples were collected in Serum Separator Tubes (Sherwood Medical, St Louis, Mo.) and then transported for processing. After processing, the serum was stored at −80° C. until use. Samples were chosen so that the overall average of the PSA for the BPH samples was similar to the average PSA level of the cancer samples. After identifying the serum samples, 39 BPH and 41 prostate cancer samples were analyzed for autoantibody reactivity to prostate cancer antigens. For clinical characteristics related to the samples, see Tables 1 and 2.

IgG Isolation and Purification

Autoantibodies were isolated from patient sera through a standard serum purification process. Melon Gel IgG Purification Kits (Thermo Fisher Scientific Inc., Rockford, Ill.) were used, according to the manufacturer's directions, to retain most serum proteins, while isolating the IgG from 50 uL aliquots of individual patient serum. After isolating the autoantibodies, their concentration was assayed to ensure a consistent amount of antibodies were dye-labeled and applied to each microarray. IgG concentration was determined by spectrometry using a BCA Protein Assay Kit (Thermo Fisher Scientific Inc., Rockford, Ill.). Each sample was then diluted or concentrated as needed to produce a lug/uL mix of IgG and buffer. 100 ug of patient serum were dye-labeled using green fluorescing Cy3 maleimide mono-reacting dye (GE Healthcare, Piscataway, N.J.) following the manufacturer's instructions. Excess dye was removed by Zeba Desalting Spin Desalting Columns (Thermo Fisher Scientific Inc., Rockford, Ill.) as described by the manufacturer's instructions.

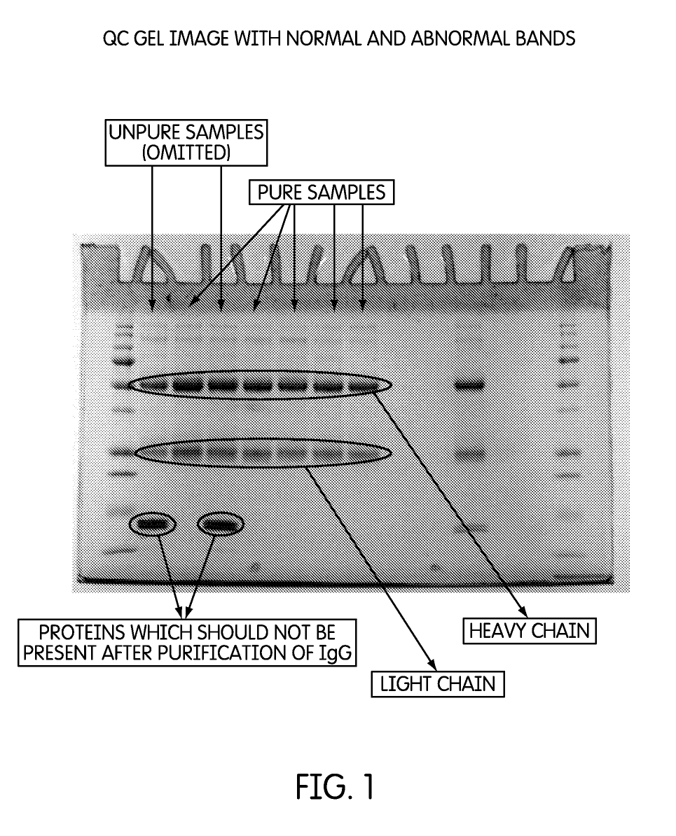

Sample purity was then tested by running each sample on an 8-16% Tris-HCl Criterion Precast Gel (Bio-Rad Laboratories, Hercules, Calif.). If a sample produced bands other than those expected for the light chain and heavy chain of the IgG, the sample was not considered in the analysis. FIG. 1 is an image of a QC gel with normal and abnormal bands.

Reverse Capture Microarray and Protocol

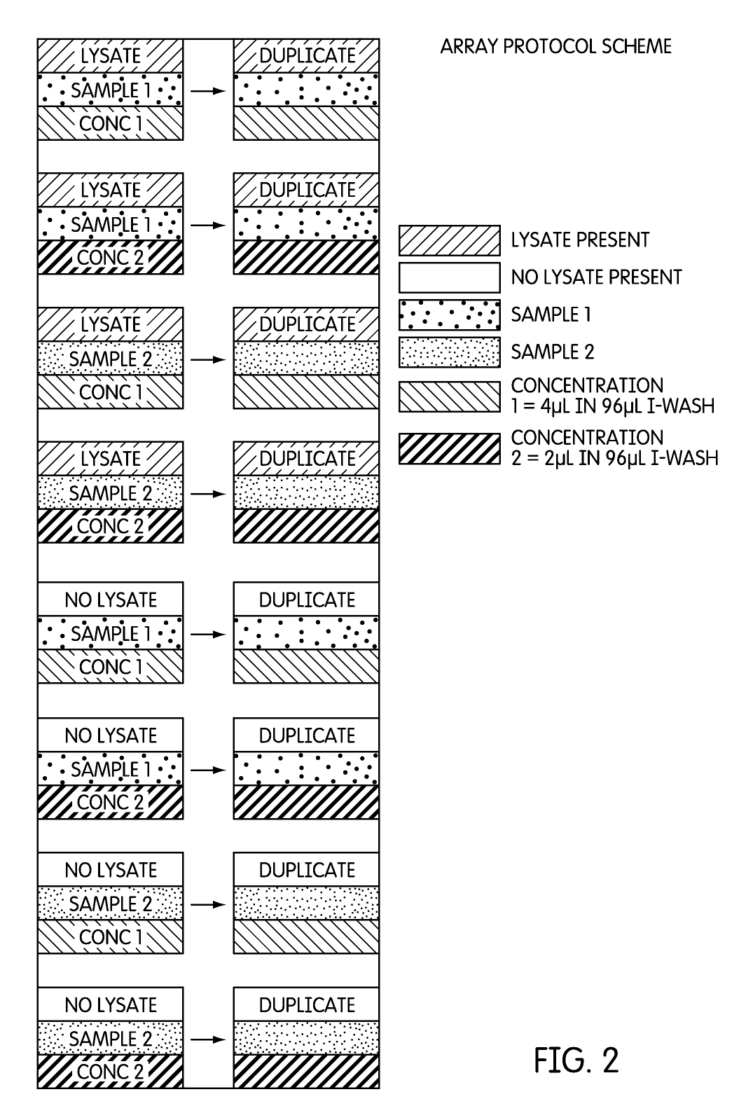

27-plex reverse capture microarrays were constructed with the antigens listed in Table 3. The arrays were first fitted with gaskets which separate the 16 individual arrays on each slide. The slides, with the gaskets, were then put into a bracket which holds the gasket firmly in place making a watertight seal. Once the slides were secured in place, 200 uL of I-block (Inanovate Inc., Raleigh, N.C.) were added to each subarray, and the platform gently rocked for thirty minutes. The blocking solution was removed and 6.25 uL of a 1 ug/uL mix of the LNCaP/PC3 cell lysate was combined with 93.75 uL of I-Wash (Inanovate Inc., Raleigh, N.C.) and added to each well. After two hours of gentle rocking while covered with tin foil, each well was thoroughly washed using a plate-washer filled with an I-Wash solution. Following the wash cycle, the Cy3 dye-labeled patient serum was added to wells according to a predetermined layout. Each row of the array was for one sample at one concentration, therefore generating two data sets for each sample at each concentration. The first concentration was 4 uL of lug/uL of Cy3 dye-labeled patient IgG mixed with 96 uL of I-Wash. The second concentration was 2 uL of lug/uL of Cy3 dye-labeled patient IgG in 98 uL of I-Wash and for each sample, the first row was used for concentration one, while the second row was used for concentration two. After incubating for one more hour, while covered with tin foil and gently rocking, the slides were put into the plate-washer and each well was rinsed with an I-Wash solution. The bracket was disassembled, the gaskets removed, and the slides centrifuged dry for 20 minutes at room temperature at 1000 rpm. A schematic of the array protocol is shown in FIG. 2.

Image Scanning and Data Collection

A PerkinElmer ScanArray 4000XL scanner and ScanArray Express software (PerkinElmer Inc., Waltham, Mass.) were used to scan each slide for fluorescence and to generate Tiff images. The Tiff image was then uploaded into GenePix Pro 6.0 (Molecular Devices, Sunnyvale, Calif.) where the data was collected and organized. GenePix Results (GPR) files were then uploaded into the bioinformatics software Acuity 4.0 (Molecular Devices, Sunnyvale, Calif.) where the data was ordered, statistically analyzed, and hierarchically clustered. Typically, before statistically analyzing and clustering the datasets, the slides would have been normalized. However, by only using one dye, any possible type of dye bias was avoided and therefore normalization was unnecessary.

Receiver Operator Characteristics Curve and Area Under the Curve Application:

Statistical Analysis Software (SAS) 9.1 (SAS Institute Inc., Cary N.C.) was used to generate receiver operator characteristics curves (ROC), which were then used to generate area under the curve (AUC) values for each arrayed autoantibody. The curve was based on the fluorescence values for each specific autoantibody from all of the patients, cancer and BPH. After arranging the values from highest to lowest for a particular autoantibody, the intensity of each fluorescence value was plotted on a sensitivity vs. 1-specificity graph. From this curve, the area under the curve was calculated, which is representative of the predictive power of the autoantibody to distinguish between cancer and BPH.

### Results

Preferential Reactivity of Autoantibodies From Sera of Patients with Prostate Cancer Versus Patients with BPH:

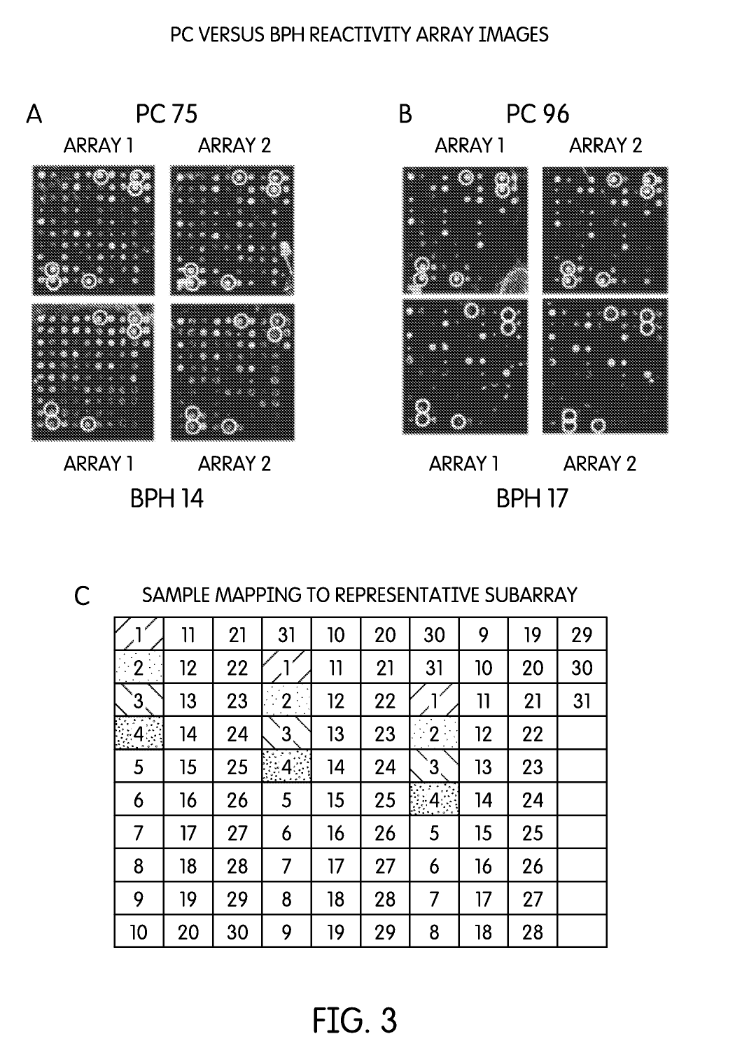

We tested the feasibility of autoantibody profiling as a potential strategy to distinguish age-matched prostate cancer from BPH patients with similar serum PSA levels, i.e., mean serum PSA for BPH is 4.1 ng/mL, and mean serum PSA for prostate cancer is 4.2 ng/mL with a Gleason score of 6 or 7 (see Tables 1 and 2). To establish our control group, the BPH samples were histologically confirmed and had a mean follow-up time of 6.56 years to rule-out a diagnosis of cancer. Results show that there is clearly preferential autoantibody reactivity with the immobilized antigens between patients with prostate cancer, as illustrated in Table 4 and FIG. 3. Table 3 is a legend to the array key in FIG. 3C and contains the actual antibody name abbreviations and unique identifying Swiss Protein Accession Numbers (abbreviated “Swiss-Prot,” the associated amino acid sequences are also included in Table 7). Since the antigens were immobilized with known monoclonal antibodies on the array, the antigens recognized by the autoantibodies were easily identified.

ROC Curve and Area Under the Curve Characteristics:

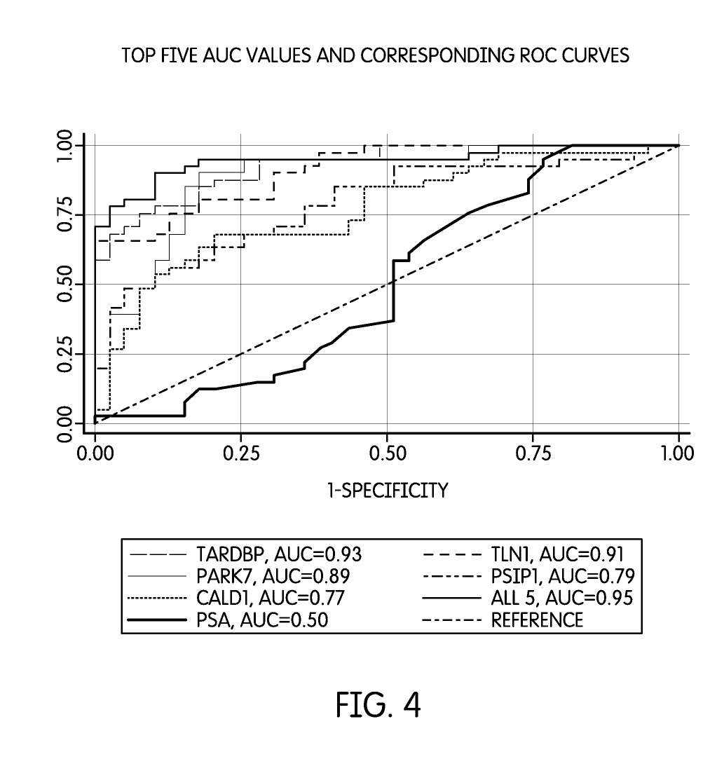

A receiver operating characteristics (ROC) curve was constructed for each of the antigens using individual fluorescence intensity values for each case and control. The top 5 antigens are shown in FIG. 4. Also included in FIG. 4 is the area under the curve (AUC) for each of the top 5 antigens. A complete list of the performance characteristics for the antigens, with their respective area under the curve, is shown in Table 4.

Reproducibility of the Microarrays:

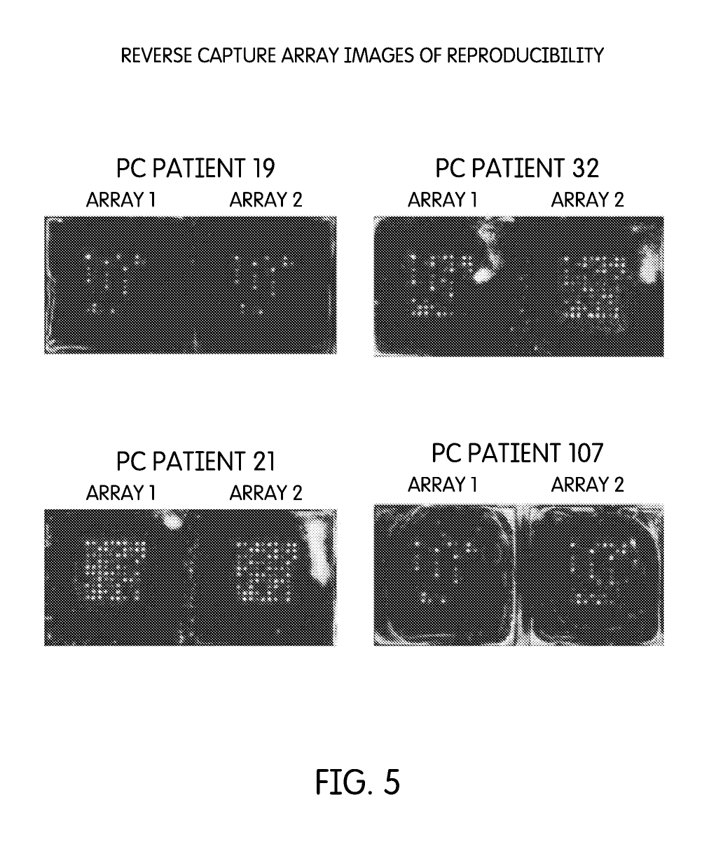

FIG. 5 illustrates the reproducibility of our customized “reverse capture” microarray. The images show the antigen-autoantibody reactivity of the same patient in two separate array runs. Although different patients may have different antigen-autoantibody reactivity, note the similarity of the reactivity in the duplicate runs (Array 1 vs. Array 2 for the same patient).

Coefficient of Variance and Platform Characteristics:

Coefficient of Variation (CV) data between antibody spots and from spot to spot across prostate cancer arrays is displayed in Table 5. Spot to spot CV data for the three replicates in each subarray and for both the subarray and its duplicate are presented. The range for each CV value is presented alongside its mean value and the averages for range and mean CV values are located at the bottom of the table. Similarly, the CV data for the BPH arrays is presented in Table 6.

TABLE 1
Prostate Cancer Patient Clinical Data/Characteristics.

| Sample number | Age | | | Follow Up |
|---|---|---|---|---|
| (internal code) | (years) | PSA (ng/mL) | Gleason score | (years) |
| 1 (PC19) | 61 | 4.2 | 3 + 3 6 | 7 |
| 2 (PC20)* | 58 | 4.7 | 3 + 3 6 | 7 |
| 3 (PC21) | 58 | 4.2 | 3 + 3 7 | 7 |
| 4 (PC32) | 57 | 5 | 3 + 3 6 | 7 |
| 5 (PC33) | 52 | 3.8 | 3 + 4 7 | 7 |
| 6 (PC34) | 56 | 4.3 | 3 + 3 6 | 7 |
| 7 (PC42) | 57 | 4.7 | 3 + 3 6 | 7 |
| 8 (PC48) | 56 | 2.4 | 3 + 3 6 | 7 |
| 9 (PC50) | 56 | 5.5 | 3 + 3 6 | 7 |
| 10 (PC54) | 53 | 2.8 | 3 + 3 6 | 7 |
| 11 (PC59) | 49 | 4.2 | 4 + 3 7 | 7 |
| 12 (PC63) | 56 | 4.8 | 4 + 3 7 | 7 |
| 13 (PC75) | 58 | 4.3 | 3 + 4 7 | 7 |
| 14 (PC78) | 51 | 2.3 | 3 + 3 6 | 7 |
| 15 (PC79) | 57 | 4.9 | 3 + 4 7 | 7 |
| 16 (PC81) | 55 | 4.6 | 3 + 3 6 | 7 |
| 17 (PC82) | 46 | 3.9 | 3 + 3 6 | 7 |
| 18 (PC85) | 56 | 2.8 | 3 + 3 6 | 7 |
| 19 (PC87) | 61 | 5 | 3 + 3 6 | 7 |
| 20 (PC88) | 56 | 3.8 | 3 + 3 6 | 6 |
| 21 (PC89) | 69 | 5.3 | 4 + 3 7 | 6 |
| 22 (PC90) | 52 | 4.8 | 3 + 3 6 | 7 |
| 23 (PC91) | 48 | 5.6 | 3 + 4 7 | 7 |
| 24 (PC92) | 54 | 0.9 | 3 + 3 6 | 6 |
| 25 (PC94) | 53 | 4.3 | 3 + 4 7 | 7 |
| 26 (PC96) | 53 | 4 | 4 + 5 9 | 7 |
| 27 (PC101) | 62 | 5.2 | 3 + 4 7 | 6 |
| 28 (PC104) | 49 | 3.6 | 3 + 3 6 | 6 |
| 29 (PC107) | 56 | 3.3 | 3 + 3 6 | 6 |
| 30 (PC115) | 60 | 5.3 | 3 + 4 7 | 6 |
| 31 (PC116) | 69 | 4.8 | 3 + 4 7 | 6 |
| 32 (PC117) | 46 | 4.3 | 3 + 3 6 | 6 |
| 33 (PC122) | 47 | 3.1 | 3 + 3 6 | 6 |
| 34 (PC131) | 66 | 4.8 | 3 + 3 6 | 6 |
| 35 (PC134) | 56 | 5.4 | 4 + 3 7 | 6 |
| 36 (PC135) | 65 | 4.1 | 3 + 4 7 | 6 |
| 37 (PC139) | 55 | 5.2 | 3 + 3 6 | 6 |
| 38 (PC142) | 58 | 3.7 | 3 + 4 7 | 6 |
| 39 (PC149) | 68 | 4.2 | 3 + 3 6 | 6 |
| 40 (PC151) | 55 | 4.2 | 3 + 4 7 | 6 |
| 41 (PC155) | 59 | 4 | 5 + 4 9 | 6 |
| Mean | 56.3 | 4.2 | 6.53 | 6.56 |

Listed in the table are the patient ID number, age, PSA level, Gleason score, and follow-up time for each PC patient sample.
The mean value for each characteristic is displayed at the bottom of the table. 41 prostate samples were used.

TABLE 2
BPH Patient Clinical Characteristics.

| Sample number | | | |
|---|---|---|---|
| (internal code) | Age (years) | PSA (ng/mL) | Follow Up (years) |
| 1 (BPH2) | 64 | 2.8 | 8 |
| 2 (BPH9) | 53 | 3.6 | 8 |
| 3 (BPH10) | 55 | 7 | 8 |
| 4 (BPH11) | 74 | 5 | 7 |
| 5 (BPH12) | 77 | 1.2 | 8 |
| 6 (BPH13) | 56 | 4.5 | 7 |
| 7 (BPH14) | 67 | 3.6 | 8 |
| 8 (BPH15) | 71 | 3.6 | 8 |
| 9 (BPH17) | 57 | 5.8 | 8 |
| 10 (BPH20) | 65 | 4.8 | 8 |
| 11 (BPH25) | 87 | 3 | 7 |
| 12 (BPH26) | 56 | 5.7 | 7 |
| 13 (BPH29) | 67 | 3.2 | 7 |
| 14 (BPH31) | 68 | 4.8 | 6 |
| 15 (BPH33) | 59 | 5.3 | 7 |
| 16 (BPH35) | 72 | 6.3 | 5 |
| 17 (BPH47) | 70 | 5 | 5 |
| 18 (BPH48) | 64 | 5.6 | 5 |
| 19 (BPH49) | 79 | 4.1 | 5 |
| 20 (BPH59) | 68 | 4.7 | 6 |
| 21 (BPH61) | 76 | 3.9 | 5 |
| 22 (BPH65) | 74 | 3 | 6 |
| 23 (BPH69) | 68 | 1.9 | 6 |
| 24 (BPH70) | 63 | 1.9 | 4 |
| 25 (BPH72) | 64 | 1.1 | 7 |
| 26 (BPH73) | 60 | 4.5 | 6 |
| 27 (BPH75) | 64 | 3.1 | 7 |
| 28 (BPH76) | 79 | 4.1 | 6 |
| 29 (BPH85) | 61 | 1.8 | 7 |
| 30 (BPH88) | 59 | 5.9 | 7 |
| 31 (BPH89) | 60 | 1.9 | 7 |
| 32 (BPH91) | 87 | 6.8 | 7 |
| 33 (BPH98) | 69 | 7 | 6 |
| 34 (BPH99) | 69 | 6.7 | 6 |
| 35 (BPH102) | 63 | 3.1 | 7 |
| 36 (BPH112) | 48 | 2 | 7 |
| 37 (BPH121) | 58 | 4 | 5 |
| 38 (BPH123) | 54 | 4.1 | 6 |
| 39 (BPH129) | 74 | 3.1 | 6 |
| Mean | 66 | 4.1 | 6.56 |

Displayed are the patient ID number, age, PSA level, and follow-up year for each BPH patient sample.
The mean value for each characteristic is displayed at the bottom of the table.
39 BPH samples were used.

TABLE 3
Spot ID for FIG. 3C and Tables 5 and 6, and Swiss-Prot
Accession Numbers (Amino Acid Sequences for antigens
recognized by antibodies are listed in Table 7).

| | Antibody Name | Swiss Accession Number |
|---|---|---|
| 1 | Sig_Con | Not applicable |
| 2 | Neg_Con | Not applicable |
| 3 | Samp_Con_H | Not applicable |
| 4 | Samp_Con_L | Not applicable |
| 5 | PARK7 | Q99497 |
| 6 | CCNA2 | P20248 |
| 7 | ACID2 | Q86YD1 |
| 8 | CALD1 | Q05682 |
| 9 | CHD-3 | Q12873 |
| 10 | DBN1 | Q16643 |
| 11 | GAPDHS | O14556 |
| 12 | HSF4 | Q9ULV5 |
| 13 | KLK3 | P07288 |
| 14 | NFAT1 | Q13469 |
| 15 | PEX10 | O60683 |
| 16 | SOS1 | Q07889 |
| 17 | STAT2 | P52630 |
| 18 | STX6 | O43752 |
| 19 | TARDBP | Q13148 |
| 20 | TLN1 | Q9Y490 |
| 21 | AR | P10275 |
| 22 | b-Tubulin | Q13885 |
| 23 | CASP8 | Q14790 |
| 24 | Cyclin D1 | P24385 |
| 25 | HSPA1A | P08107 |
| 26 | p53 | P04637 |
| 27 | p73 | O1535O |
| 28 | PSIP1 | O75475 |
| 29 | PTEN | P60484 |
| 30 | PXN | P49023 |
| 31 | TOP1 | P11387 |

Table contains the antibody name abbreviations that correspond to the antibody spot ID numbers used in FIG. 3C and Tables 5 and 6 (spot ID #'s 1 through 31).
The first four table entries are controls arrayed on each slide.
Also included are the unique Swiss Protein Accession Numbers for each antibody.

TABLE 4
AUC Values for all 27 Antibodies and PSA (Concentration 1).

| Ab Name | AUC | Rank |
|---|---|---|
| TARDBP | 0.927 | 1 |
| TLN1 | 0.911 | 2 |
| PARK7 | 0.886 | 3 |
| PSIP1 | 0.789 | 4 |
| CALD1 | 0.77 | 5 |
| p73 | 0.76 | 6 |
| PTEN | 0.675 | 7 |
| PXN | 0.667 | 8 |
| PEX10 | 0.667 | 9 |
| KLK3 | 0.606 | 10 |
| DBN1 | 0.6 | 11 |
| NFAT1 | 0.592 | 12 |
| B Tubulin | 0.591 | 13 |
| SOS1 | 0.583 | 14 |
| HSF4 | 0.581 | 15 |
| TOP1 | 0.567 | 16 |
| HSPA1A | 0.567 | 17 |
| ACID2 | 0.556 | 18 |
| STAT2 | 0.54 | 19 |
| p53 | 0.512 | 20 |
| CHD 3 | 0.506 | 21 |
| CASP8 | 0.498 | 22 |
| STX6 | 0.498 | 23 |
| AR | 0.478 | 24 |
| GAPDHS | 0.476 | 25 |
| Cyclin D1 | 0.465 | 26 |
| CCNA2 | 0.438 | 27 |
| Serum level PSA (ng/ml) | 0.50 | |

This table displays the AUC values for all antibodies tested on the 27-plex microarray platform. They are ranked according to AUC values for concentration 1, which is 4 ul of 1 ug/ul of Cy3 dye-labeled patient IgG mixed with 96 ul of I-wash buffer.
AUC for PSA is calculated from the sample's serum PSA level.

TABLE 5
Coefficient of Variation Data for PC Arrays.

| | Well 1 | | | Well 2 | | | Well to Well | | |
|---|---|---|---|---|---|---|---|---|---|
| | | | Spot to | | | Spot to | | | Well to |
| | | | Spot CV | | | Spot CV | Well to Well | | Well CV |
| Spot # | CV Range | | Average | CV Range | | Average | Range | | Average |
| 1 | 0% | 32% | 12% | 2% | 40% | 16% | 0% | 25% | 9% |
| 2 | 3% | 82% | 18% | 3% | 48% | 20% | 1% | 64% | 17% |
| 3 | 1% | 36% | 11% | 2% | 45% | 17% | 2% | 47% | 13% |
| 4 | 0% | 36% | 13% | 2% | 77% | 18% | 0% | 49% | 14% |
| 5 | 1% | 63% | 11% | 1% | 34% | 8% | 0% | 58% | 12% |
| 6 | 2% | 44% | 13% | 3% | 33% | 13% | 0% | 83% | 18% |
| 7 | 2% | 59% | 13% | 1% | 143% | 17% | 1% | 92% | 19% |
| 8 | 0% | 69% | 14% | 2% | 117% | 16% | 0% | 81% | 17% |
| 9 | 2% | 78% | 19% | 1% | 49% | 14% | 0% | 68% | 16% |
| 10 | 1% | 105% | 18% | 3% | 98% | 19% | 1% | 91% | 21% |
| 11 | 1% | 95% | 18% | 4% | 39% | 17% | 0% | 76% | 17% |
| 12 | 2% | 47% | 17% | 3% | 53% | 16% | 0% | 69% | 17% |
| 13 | 1% | 49% | 15% | 1% | 74% | 16% | 0% | 85% | 18% |
| 14 | 2% | 43% | 13% | 1% | 76% | 16% | 0% | 81% | 16% |
| 15 | 3% | 37% | 14% | 1% | 62% | 14% | 0% | 85% | 19% |
| 16 | 3% | 41% | 18% | 4% | 74% | 20% | 2% | 85% | 24% |
| 17 | 1% | 62% | 14% | 3% | 46% | 14% | 0% | 95% | 21% |
| 18 | 2% | 100% | 17% | 3% | 121% | 26% | 1% | 104% | 27% |
| 19 | 2% | 37% | 14% | 1% | 38% | 14% | 0% | 59% | 11% |
| 20 | 2% | 48% | 10% | 1% | 41% | 9% | 0% | 44% | 12% |
| 21 | 2% | 114% | 20% | 2% | 114% | 16% | 0% | 90% | 21% |
| 22 | 2% | 41% | 8% | 3% | 85% | 12% | 0% | 69% | 15% |
| 23 | 1% | 44% | 12% | 2% | 32% | 12% | 1% | 77% | 18% |
| 24 | 1% | 36% | 10% | 2% | 100% | 15% | 0% | 74% | 21% |
| 25 | 1% | 92% | 14% | 1% | 63% | 14% | 1% | 77% | 20% |
| 26 | 1% | 69% | 12% | 2% | 125% | 18% | 0% | 88% | 19% |
| 27 | 3% | 85% | 14% | 3% | 69% | 13% | 0% | 114% | 19% |
| 28 | 1% | 128% | 16% | 2% | 64% | 15% | 0% | 96% | 22% |
| 29 | 4% | 38% | 18% | 3% | 54% | 20% | 1% | 70% | 16% |
| 30 | 0% | 39% | 14% | 2% | 62% | 17% | 1% | 62% | 19% |
| 31 | 1% | 51% | 9% | 1% | 36% | 10% | 0% | 56% | 16% |
| Mean | 2% | 61% | 14% | 2% | 68% | 16% | 0% | 75% | 17% |

Displayed is each antibody (spot ID #), its average CV data from spot to spot in both sample's arrays, the ranges for CV values in both wells, the average CV data from well to well, and the CV ranges for the well to well data. At the bottom of the table, the mean value for all antibody CV data including ranges and well to well data is displayed.

TABLE 6
Coefficient of Variation Data for BPH Arrays.

| | Well 1 | | | Well 2 | | | Well to Well | | |
|---|---|---|---|---|---|---|---|---|---|
| | | | Spot to | | | Spot to | | | Well to |
| | | | Spot CV | | | Spot CV | Well to Well | | Well CV |
| Spot # | CV Range | | Average | CV Range | | Average | Range | | Average |
| 1 | 3% | 25% | 8% | 1% | 19% | 9% | 0% | 21% | 6% |
| 2 | 4% | 37% | 19% | 1% | 62% | 23% | 2% | 46% | 13% |
| 3 | 2% | 42% | 14% | 1% | 51% | 14% | 0% | 78% | 12% |
| 4 | 4% | 49% | 15% | 2% | 105% | 21% | 1% | 50% | 13% |
| 5 | 5% | 37% | 15% | 2% | 57% | 22% | 1% | 72% | 14% |
| 6 | 1% | 53% | 14% | 2% | 43% | 14% | 0% | 88% | 15% |
| 7 | 1% | 83% | 15% | 2% | 70% | 14% | 0% | 73% | 14% |
| 8 | 2% | 55% | 13% | 0% | 70% | 16% | 0% | 88% | 17% |
| 9 | 1% | 41% | 16% | 4% | 71% | 20% | 1% | 35% | 11% |
| 10 | 2% | 62% | 20% | 1% | 72% | 18% | 0% | 37% | 14% |
| 11 | 3% | 40% | 16% | 4% | 86% | 21% | 1% | 51% | 12% |
| 12 | 1% | 160% | 17% | 3% | 79% | 21% | 1% | 110% | 16% |
| 13 | 3% | 65% | 14% | 2% | 91% | 17% | 0% | 67% | 13% |
| 14 | 1% | 121% | 16% | 3% | 75% | 18% | 1% | 65% | 15% |
| 15 | 3% | 42% | 14% | 4% | 76% | 15% | 0% | 99% | 14% |
| 16 | 2% | 32% | 13% | 4% | 40% | 16% | 0% | 100% | 14% |
| 17 | 2% | 34% | 11% | 2% | 81% | 19% | 0% | 88% | 16% |
| 18 | 1% | 122% | 20% | 2% | 94% | 25% | 0% | 83% | 21% |
| 19 | 4% | 56% | 21% | 3% | 75% | 21% | 0% | 74% | 13% |
| 20 | 3% | 35% | 15% | 2% | 91% | 17% | 0% | 67% | 12% |
| 21 | 2% | 56% | 16% | 4% | 66% | 18% | 1% | 40% | 13% |
| 22 | 1% | 33% | 10% | 2% | 44% | 13% | 0% | 46% | 10% |
| 23 | 2% | 113% | 14% | 2% | 69% | 16% | 0% | 68% | 13% |
| 24 | 3% | 54% | 12% | 1% | 84% | 16% | 0% | 63% | 12% |
| 25 | 3% | 53% | 15% | 2% | 52% | 18% | 0% | 64% | 12% |
| 26 | 2% | 40% | 13% | 4% | 67% | 19% | 1% | 74% | 16% |
| 27 | 1% | 48% | 16% | 1% | 68% | 21% | 0% | 74% | 17% |
| 28 | 2% | 44% | 15% | 4% | 66% | 19% | 0% | 89% | 18% |
| 29 | 9% | 66% | 30% | 4% | 105% | 28% | 2% | 71% | 16% |
| 30 | 2% | 46% | 23% | 3% | 95% | 24% | 0% | 73% | 16% |
| 31 | 2% | 46% | 13% | 2% | 39% | 14% | 0% | 30% | 10% |
| Mean | 2% | 58% | 16% | 2% | 70% | 18% | 0% | 67% | 14% |

Presented is each antibody (spot ID #), its average CV data from spot to spot in both sample's arrays, the ranges for CV values in both wells, the average CV data from well to well, and the CV ranges for the well to well data. At the bottom of the table, the mean value for all antibody CV data including ranges and well to well data is displayed.

### APPENDIX

Table 7 correlates specific protein sequences with identification information used in the present application. The first column lists a SEQ ID NO which corresponds to the amino acid sequence of the protein named in the last column. The sequence for each protein is shown in the attached Sequence Listing. The name of the gene encoding the protein is shown in the second column and the accession number for the protein in the Swiss Prot database (also showing the protein sequence) is shown as the third column. In all cases the named proteins and genes are human.

TABLE 7
Biomarker Identification Information

| Seq | | Swiss Prot | |
|---|---|---|---|
| ID | | Accession | |
| NO: | Gene | No. | Protein Name |
| 1 | KLK3 | P07288 | Prostate-specific antigen |
| 2 | DBN1 | Q16643 | Drebrin |
| 3 | p53 | P04637 | Cellular tumor antigen p53 |
| 4 | SOS1 | Q07889 | Son of sevenless homolog 1 |
| 5 | GAPDHS | O14556 | Glyceraldehyde-3-phosphate |
| | | | dehydrogenase, testis-specific |
| 6 | CALD1 | Q05682 | Caldesmon |
| 7 | HSPA1A | P08107 | Heat shock 70 kDa protein 1A/1B |
| 8 | CCND1 | P24385 | G1/S-specific cyclin-D1 |
| | (cyclin-D1) | | |
| 9 | TBB2A | Q13885 | Tubulin beta-2A chain |
| | (B-Tubulin) | | |
| 10 | TOP1 | P11387 | DNA topoisomerase 1 |
| 11 | AR | P10275 | Androgen receptor |
| 12 | CASP8 | Q14790 | Caspase-8 |
| 13 | CCNA2 | P20248 | Cyclin-A2 |
| 14 | PARK7 | Q99497 | Protein DJ-1 |
| 15 | PEX10 | O60683 | Peroxisome biogenesis factor 10 |
| 16 | PSIP1 | O75475 | PC4 and SFRS1-interacting protein |
| 17 | PTEN | P60484 | Phosphatidylinositol-3,4,5-trisphosphate |
| | | | 3-phosphatase and dual-specificity |
| | | | protein phosphatase PTEN |
| 18 | PXN | P49023 | Paxillin |
| 19 | TARDBP | Q13148 | TAR DNA-binding protein 43 |
| 20 | TLN1 | Q9Y490 | Talin-1 |
| 21 | CHD-3 | Q12873 | Chromodomain-helicase-DNA-binding |
| | (CHD 3) | | protein 3 |
| 22 | NFAT1 | Q13469 | Nuclear factor of activated T-cells, |
| | (NFATC2) | | cytoplasmic 2 |
| 23 | STX6 | O43752 | Syntaxin-6 |
| 24 | p73 | O15350 | Tumor protein p73 |
| 25 | HSF4 | Q9ULV5 | Heat shock factor protein 4 |
| 26 | STAT2 | P52630 | Signal transducer and activator of |
| | | | transcription 2 |
| 27 | PTOV1 | Q86YD1 | Prostate tumor-overexpressed gene 1 |
| | (ACID2) | | protein |

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A method of diagnostically evaluating a subject for prostate cancer, comprising:
a) obtaining a test biological sample from a said subject;
b) assaying said test biological sample for two or more tumor associated markers (TAMs), wherein said two or more TAMs are selected from a group consisting of: TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, CCNA2;
c) comparing the results obtained in step b) with an assay of said two or more TAMs in a control sample or to predetermined TAM concentration levels or ratios thereof; and
d) concluding that said subject is at increased risk of having prostate cancer if the amount of said one or more TAMs in said test biological sample is higher than in said control sample or is different by a predetermined amount to predetermined concentration levels or ratios thereof.

2. The method of claim 1, wherein at least 5 different TAMs selected from a group containing TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, CCNA2 are assayed.

3. (canceled)

4. The method of claim 1, wherein said test biological sample is blood, plasma, or serum.

5. The method of claim 1, wherein said test biological sample is urine, saliva, prostate fluid or prostate tissue.

6. The method of claim 1, wherein the assay of said one or more TAMs is an ELISA, radioimmunoassay, immunoassay, radioreceptor assay, bead based assay, label free assay, reverse capture assay, flow based assay or any other assay known to those skilled in the art.

7. The method of claim 1, wherein the assay of said one or more TAMs is an antibody profiling assay.

8. The method of claim 1, wherein said control sample is from a patient with benign prostate hyperplasia.

9. A method of diagnostically evaluating a subject for prostate cancer, comprising:
a) obtaining a test biological sample from a said subject;
b) assaying said test biological sample for two or more autoantibody biomarkers, wherein said one or more autoantibodies are selected from a first group consisting of autoantibodies to: TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, CCNA2
c) comparing the results obtained in step b) with an assay of said two or more autoantibodies in a control sample or to predetermined autoantibody concentration levels or ratios thereof; and
d) concluding that said subject is at increased risk of having prostate cancer if the amount of said one or more autoantibodies in said test biological sample is higher than in said control sample or is different by a predetermined amount to predetermined concentration levels or ratios thereof.

10. The method of claim 9, wherein at least 5 different TAMs selected from a group containing TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, CCNA2 are assayed.

11. (canceled)

12. The method of claim 9, wherein said test biological sample is blood, plasma, serum, urine, saliva, prostate fluid or prostate tissue.

13. (canceled)

14. The method of claim 9, wherein the assay of said one or more TAMs is an ELISA, radioimmunoassay, immunoassay, radioreceptor assay, bead based assay, label free assay, reverse capture assay, flow based assay or any other assay known to those skilled in the art.

15. The method of claim 9, wherein the assay of said one or more TAMs is an antibody profiling assay.

16. The method of claim 9, wherein said control sample is from a patient with benign prostate hyperplasia.

17-27. (canceled)

28. A composition that can be used in diagnostically evaluating a subject for prostate cancer, comprising:
a) a solid support wherein said solid support is selected from the group consisting of: a glass, plastic, carbon, polymer based, metallic or silicon plate, slide, chip or cartridge;
b) at least 2 different TAMs selected from the group consisting of: TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, CCNA2, wherein each different TAM is attached to a different site on said solid support.

29. The composition of claim 28, wherein at least 5 TAMs are attached to said solid support, and said at least 5 TAMs are selected from the group consisting of: TARDBP, TLN1, PARK7, PSIP1, CALD1, p73, PTEN, PXN, PEX10, KLK3, DBN1, NFAT1, B-Tubulin, SOS1, HSF4, TOP1, HSPA1A, ACID2, STAT2, p53, CHD 3, CASP8, STX6, AR, GAPDHS, Cyclin D1, and CCNA2.

30. The composition of claim 28, wherein all 27 TAMs are attached to said solid support.

31. The composition of claim 28, wherein each TAM is directly attached to said solid support by a monoclonal antibody that specifically recognizes said TAM.

32. The composition of claim 28, wherein each TAM is directly attached to said solid support by an antibody fragment that specifically recognizes said TAM.

33. The composition of claim 28, wherein each TAM is directly attached to said solid support by an aptamer or other capture agent that specifically recognizes said TAM.

34. The composition of claim 28, wherein each TAM is directly attached to said plate, slide, chip or cartridge following an arraying/printing step.
